(19) United States
(12) Patent Application Publication
(10) Pub. No.: US 2013/0060304 A1
(43) Pub. Date: Mar. 7, 2013

(54) Method and Apparatus for Generating Electrotherapeutic or Electrodiagnostic Waveforms

(71) Applicants: Travis Daniel LaTendresse, San Jose, CA (US); Brain D. Wichner, Otter Rock, OR (US)

(72) Inventors: Travis Daniel LaTendresse, San Jose, CA (US); Brain D. Wichner, Otter Rock, OR (US)

(73) Assignee: Brian D. Wichner, Otter Rock, OR (US)

(21) Appl. No.: 13/668,042

(22) Filed: Nov. 2, 2012

(51) Int. Cl. A61N 1/36

(52) U.S. Cl. 607/59

(57) ABSTRACT

Subject matter includes a device comprising: an input port to receive a waveform file for a waveform to be electrically applied to one or more patients via an output port; and electronics configured to: generate the waveform having a shape, magnitude, or frequency based, at least in part, on the waveform file; and provide the waveform to the output port.

### BACKGROUND

1. Field:

Subject matter disclosed herein relates to an apparatus and method for providing an electric waveform to a patient.

2. Information:

A number of techniques for treating a patient or detecting a physical condition of a patient may involve applying electrical energy via electrodes in contact with the patient. Such electrodes may comprise pads having an adhesive (or a water-activated adhesive) to temporarily affix the pads to a portion of a patient. For example, a transcutaneous electrical nerve stimulation (TENS) device may apply electric current to a patient via electrode pads to stimulate nerves of the patient for therapeutic purposes. In another example, muscle loss of a patient may be determined using electric impedance myography (EIM), which may measure resistance of a muscle to an electrical current by passing an amount of current through the muscle using electrodes.

There are various types of apparatuses for applying electrical energy to a patient. For example, an interference-type apparatus may stimulate structures located within a patient's body, such as muscles or nerves that control muscle action, which may be reached with relatively high frequency signals, but may be responsive to relatively low frequency signals. This apparatus may operate by applying two primary signals of relatively high, but slightly different, frequencies to a patient's body. The primary signals, due to their relatively high frequency, may penetrate the patient's body and reach the aforementioned structures where they combine and produce a beat signal having a relatively low frequency that is equal to the slight difference in the frequencies of the primary signals. For example, U.S. Pat. No. 4,374,524 to Hudek (1983) illustrates the use of a square-wave signal generator in conjunction with a plurality of phase-locked loops and low-pass filters to produce a plurality of sine-wave, primary signals. In other examples of interference-type apparatuses, U.S. Pat. No. 4,071,033 to Nawracaj et al. (1978) and U.S. Pat. No. 4,153,061 to Nemec (1979), in addition to providing two primary signals of different frequencies, also amplitude modulate the primary signals to achieve various therapeutic effects. For example, in Nawracaj et al., two square-wave, primary signals are amplitude modulated by either a square-wave, ramp, exponential, semi-sine or sine-wave signal. In Nemec et al., two sine-wave, primary signals are modulated by two low-frequency sine-wave signals to achieve muscle stimulation at a point of application to the patient's body in addition to producing a beat signal therein.

Another known type of apparatus for applying electrical energy to a patient's body is shown in U.S. Pat. No. 4,392,496 to Stenton (1983). Stenton applies two signals to a patient's body in an alternating fashion to achieve muscle stimulation and prevent disuse atrophy. Further, to achieve optimal muscle stimulation and enhance comfort of the patient, Stenton allows for the adjustment of several parameters associated with the applied signals, such as amplitude and frequency.

Yet another apparatus for administering an electrical stimulation to a patient's body is illustrated in U.S. Pat. No. 4,580,570 to Sarrell et al. (1986). A method of Sarrell is characterized by an application of pulses that have a relatively high voltage, high peak but low average current, and short duration. Moreover, an apparatus of Sarrell may be adjusted to apply the aforementioned pulses continuously, periodically, or in an alternating fashion.

Injured tissue may result from force transferring to an area of a patient not designed to absorb the force. An inability to absorb force properly may be due to an inability to control muscles properly. Applying electrical energy to a patient may allow a therapist to search a patient's body for a source of an injury, thus allowing the therapist to know where on the patient to perform therapy.

Applying electrical energy to a patient may increase permeability of muscle tissues of a patient. Often, injuries may not efficiently heal because blood cannot flow to an injured area. Applying electrical energy to a patient may break bonds holding scar tissue together and flush the scar tissue away with increased blood flow. With less scar tissue surrounding an injured area, more blood may be able to flow to an injury site and shorten healing time.

Rate of healing may depend on an amount of blood flow to an area of injury. Applying electrical energy to a patient may increase blood flow. Increasing blood flow may allow the body of a patient to bring more protein to an area of injury for repair and for flushing out toxins associated with inflammation and scar tissue, for example.

### BRIEF DESCRIPTION OF THE FIGURES

Non-limiting and non-exhaustive embodiments will be described with reference to the following figures, wherein like reference numerals refer to like parts throughout the various figures unless otherwise specified.

FIG. 1 is a cross-sectional schematic diagram illustrating electrodes for applying one or more electrical signals to a portion of a patient, according to an embodiment.

FIG. 2 is a schematic diagram illustrating a device, according to an embodiment.

FIG. 3 is a schematic block diagram illustrating a system for exchanging waveform files, according to an embodiment.

FIG. 4 is a schematic diagram illustrating at least a portion of a waveform file, according to an embodiment.

FIG. 5 is a schematic diagram illustrating a time sequence involving waveform file expiration, according to an embodiment.

FIGS. 6 and 7 show example waveforms plotted as magnitude of voltage or current versus time, according to embodiments.

FIGS. 8A and 8B show a first waveform and a second waveform, respectively, plotted as magnitude of voltage or current versus time, according to an embodiment.

FIG. 9 shows several waveforms for various applications, plotted as magnitude of voltage or current versus time, according to an embodiment.

FIGS. 10 and 11 show example waveforms plotted as magnitude of voltage or current versus time, according to embodiments.

FIG. 12 is a flow diagram of a process for generating a waveform, according to an embodiment.

FIG. 13 is a flow diagram of a process for ordering a waveform file, according to an embodiment.

FIG. 14 is a schematic block diagram illustrating a system for applying a waveform to a patient, according to an embodiment.

FIG. 15 is a schematic diagram illustrating an embodiment of a computing system including a memory module.

FIG. 16 is a schematic diagram of a device, according to an embodiment.

FIG. 17 is a schematic diagram of a device and amplifier device, according to an embodiment.

FIG. 18 is a schematic diagram of a device and amplifier device, according to another embodiment.

### DETAILED DESCRIPTION

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments.

Impedance may refer to the opposition that a path of electrical current presents to the passage of the current if a voltage is applied. For example, in quantitative terms, impedance may comprise a complex ratio of the voltage to the current. Impedance (e.g., for time-varying electrical signals) may comprise an extension of the concept of resistance (e.g., non-time-varying electrical signals), and may include both magnitude and phase, unlike resistance, which may only include magnitude. In situations involving time-varying electrical signals, mechanisms in addition to normal resistance (e.g., ohmic resistance for non-time-varying electrical signals) may impede flow of current. Such mechanisms may comprise induction of voltages in conductors self-induced by magnetic fields of currents (inductance), and electrostatic storage of charge induced by voltages between conductors (capacitance). Impedance based, at least in part, on these two effects may collectively be referred to as reactance and forms an imaginary part of complex impedance whereas resistance forms a real part, for example.

The terms “resistance” and “impedance” are used herein interchangeably to mean the same thing unless used in the context of a sentence that indicates otherwise. For example, “resistance” means impedance that may comprise an inductive reactance, capacitive reactance, and/or ohmic resistance. On the other hand, “impedance” may mean ohmic resistance and may or may not include inductive reactance and/or capacitive reactance. Again, a context or description of a sentence or portion of text in which such terms are used may indicate one meaning over another meaning. The term “resistance” may comprise inductive reactance, capacitive reactance, and/or ohmic resistance. If “resistance” is intended to exclude inductive reactance and/or capacitive reactance then the term “ohmic resistance” is used.

The term “patient” is recited in examples herein. A patient need not comprise a subject who is ill, sick, or stricken with any particular medical condition. A patient may comprise a medical patient, a dental patient, a physical therapy client, a massage client, or one who seeks treatment or a physical process applied to any portion of their body for any of a number of reasons. Unless otherwise described, a patient may comprise human, animal, fish, reptile, bird, and so on. In some embodiments, a patient may comprise abiotic systems or material, such as liquid, mineral, plastics, etc., although example embodiments are directed to biotic systems. For example, embodiments of techniques described may be applied in cases where a patient is human or where a patient is a fish or animal, and claimed subject matter is not limited in this respect. To describe a particular implementation, techniques may be applied to diagnose a physical condition (e.g., muscle mass, cancer, blood chemistry, and so on) of a human patient. In another implementation, techniques may be applied to perform research regarding any of a number of physical parameters of various aquatic species. In the latter implementation, the “patient” may comprise a particular aquatic specimen. Other implementation may involve animals, and so on. Accordingly, though the following descriptions may indicate a human patient, claimed subject matter is not limited in this respect. Further, “patient” need not comprise a person undergoing or seeking medical treatment or diagnoses. For example, a patient may comprise any person (e.g., or other species, as described above) to which a waveform may be applied for any reason.

Biological elements of a patient may comprise any portion or combination of portions of the patient, such as skin, muscle tissue, organs, normal or cancer cells, blood, ligaments, tendons, bones, scar tissue, and so on. Such biological elements may be microscopic or macroscopic. Such biological elements may be in any type of condition, such as healthy or normal, damaged or injured, deteriorated, inflamed, and so on.

In some embodiments, applications of electrical energy (e.g. for muscle stimulation, cellular regeneration, physical diagnosis, and so on) may involve a power source, a signal generator, at least two electrodes, and leads (e.g., cables, wires, conductors, and so on). Electrical energy application may comprise transcutaneous application, involving leads or electrodes on skin of a patient, for example. Electrical energy may comprise a waveform having a number of parameters, including one or more frequencies, voltage/current amplitude, energy, power, zero-offset, slope, and so on.

In an embodiment, a device, which may comprise a medical device, may be used to apply one or more electrical waveforms to a patient. A waveform may comprise an electronic signal that may be used for therapy, treatment, or diagnostics of one or more medical conditions of a patient. Different waveforms may have different shapes, frequencies, amplitudes, and so on. Different waveforms may be used to treat different patients, to treat different medical conditions, to perform treatment at various stages of application to a patient, to detect medical conditions of different portions of a patient, to measure or detect different medical conditions of a patient, and so on.

In another embodiment, a device may be used to apply one or more electrical waveforms to one or more solenoids, which may be positioned in contact or relatively near skin of a patient. In such a case, one or more solenoids may electromagnetically induce one or more electrical waveforms (e.g., a microcurrent waveform) in a patient. Such waveforms may comprise electronic signals that may be used for therapy, treatment, or diagnostics of one or more medical conditions of a patient. As mentioned above, different waveforms may have different shapes, frequencies, amplitudes, and so on.

Waveforms may comprise any of a number of forms. For example, a waveform may comprise digital electronic signals transmitted in a conductor or transmitted wirelessly, may comprise digital electronic signals stored in a storage medium such as a memory device, may comprise an analog electronic signal transmitted in cables or conductors (e.g., to/from a patient), may comprise a digital or analog code readable by a processor to generate a digital or analog electronic signal, and so on.

A waveform may be generated by a device based, at least in part, on a waveform file, which may include, among other things, instructions for generating the waveform. For example, a device capable of applying five different waveforms to patients may store thereon five waveform files that comprise information regarding the five different waveforms, respectively.

In an embodiment, a waveform file may comprise instructions executable by a device to generate a waveform and may allow a user operating the device to vary one or more of a number of parameters of the waveform. For example, a waveform file may comprise instructions for generating a double-exponential waveform, which may comprise two component waves: a low-frequency wave and a high-frequency wave. The instructions may allow a user to adjust controls (e.g., dials, knobs, etc.) to separately vary the frequency or amplitude of either wave. The instructions may also place constraints on values of any of a number of parameters of the waveform. For example, a user may be constrained to adjust voltage amplitude of the wave within a range of zero to 100 volts. In a particular example, a waveform file for a microcurrent waveform may place a constraint on current output generated by a device to less than several hundred micro-amps. Sensitivity of adjustment controls of a device may change based, at least in part, on instructions included in a waveform file. For example, instructions in a waveform file may set forth that one rotation of an amplitude adjusting knob may correspond to 10.0 volts for a waveform that may vary between zero and 50.0 volts. In another example, instructions in a waveform file may set forth that one rotation of an amplitude knob may correspond to 1.0 volts for a waveform that may vary between zero and 5.0 volts. Of course claimed subject matter is not limited to such numerical examples.

In some embodiments, waveform files may be sold, purchased, or traded, exchanged, and so on. In other embodiments, waveform files may comprise a commodity. Waveform files may be sold or made available by a provider or distributor. Waveform files may be purchased or received by a client or customer. Waveform files may be transferred among entities (e.g., sellers or purchasers) via the Internet (e.g., using email) or via physically transportable memory devices (e.g., flash card, disk, hard disk, and so on).

In an embodiment, a device may generate or provide an electronic signal comprising a waveform at an output port. Such a waveform may be applied via electrodes to a patient. The electronic signal may be generated based, at least in part, on a waveform file executed by a processor of a device. A waveform file may be resident on a device, downloaded to the device, or temporarily stored on the device. Any of a number of waveforms may be generated by a device since any of a number of waveform files may be executed by the device. This provides a number of advantages. For example, a device need not be limited to generating or providing merely a single or few waveforms, but instead may have a capability to generate or provide an unlimited number of waveforms at any time. Accordingly, a device need not be “hard-wired” to generate and output a single waveform, wherein merely an amplitude or frequency of the waveform may be adjustable but the shape of the waveform may not be adjustable.

A device may include one or more input ports to receive a waveform file from a source external to the device. For example, a source may be considered external to a device if the source is located outside an enclosure of a device. Such sources may comprise a portable memory device, a server on the Internet, a wireless transmitter located outside an enclosure of the device, and so on. An input port may comprise a universal serial bus (USB) port or Internet connection, for example. A device may further comprise electronics to generate a waveform having a shape, magnitude, or frequency based, at least in part, on the waveform file, and to provide the waveform to an output port (e.g., and subsequently to a patient). Such a device may further comprise electronics to generate a waveform having a shape, magnitude, or frequency based, at least in part, on the waveform file and on patient information regarding one or more patients, wherein the patient information comprises information of a patient and one or more of their: body weight, age, sex, heart condition, injury status, injury/health history, and history of treatment using the device. Patient information may be stored in a memory of a device or accessible by a processor of the device that executes or reads a waveform file, for example.

In one implementation, a waveform file for a waveform may comprise an expirable waveform file that expires after a predetermined time that the waveform is applied to one or more patients. A waveform file may further comprise a time code that indicates an expiration time of an expirable waveform file. In such a case, a device may further comprise a clock to measure an elapsed time that the waveform is applied to one or more patients, means for determining whether the elapsed time exceeds the expiration time, and a user interface (e.g., a display) to indicate if the expirable waveform file is expired. For example, such means may comprise a processor executing code or electronic components configured to perform the means. Such a user interface or input port may allow a user to enter or download code to instruct a device to extend an expiration time, for example.

In one implementation, a waveform file may comprise code representative of one or more waveforms and one or more protocols comprising instructions about how the one or more waveforms are to be generated. Such instructions may further generate, at least in part, output for a display of a device that may be observed by a patient or user of the device. For example, instructions for treatment of a patient for a particular muscle condition may set forth that a device is to generate a first waveform at first peak voltage and a first frequency for three minutes, and to display a timer countdown or waveform description in large display font. The instructions may further set forth that the peak voltage of the first waveform is to be increased to a second peak voltage over a period of 20 seconds and then held constant for three minutes, and to display a timer countdown or waveform description in large display font. The instructions may further set forth that the frequency of the first waveform is to be increased to a second frequency over a period of 5 seconds, the peak voltage ramped up to a third peak voltage over one minute, and then held constant for three minutes, and to display a timer countdown or waveform description in large display font. The instructions may further set forth that the first waveform is to be replaced by a second waveform at a fourth peak voltage then held constant for ten minutes, and to display a timer countdown or waveform description in large display font. And so on. The instructions may further set forth that the generated output from the device be reduced to zero intensity (e.g., volts or current). Of course, particular numerical values are merely example, and claimed subject matter is not so limited.

Waveform files that include instruction protocols may provide a number of advantages, such as providing a technique for patients to treat themselves while a device automatically operates the characteristics (e.g., intensity, frequency, waveshape, and so on) of one or more waveforms. In one implementation, such instruction protocols may be designed differently for different patients having different conditions. In another implementation, such instruction protocols may be adjustable for particular patients based, at least in part, on information regarding the particular patients. For example, based, at least in part, on a questionnaire filled out by a patient, an instruction protocol may adapt to particular conditions of the patient. In one technique, an instruction protocol may comprise code readable by a processor in a device, wherein the processor may execute the code based, at least in part, on information regarding a patient, for example. Of course, such details of a waveform file are merely examples, and claimed subject matter is not so limited.

In an embodiment, a method performed by a waveform file provider may comprise receiving an order from a client for a waveform file for a waveform to be applied by a device to one or more patients, and transmitting the waveform file electronically (e.g., via the Internet) to the client. In a particular implementation, such a method may be computer-implemented, that is, performed by a process of executing computer-readable code. In such a case, for example, an order from a client may be transmitted electronically and received via an Internet or other connection. A waveform file may be transmitted to the client electronically over the Internet or other connection, for example. In an alternative embodiment, a waveform file may be sent to a client via a transportable memory device, such as a flash drive, memory disk, and so on. In such a case, for example, a computer display or print-out may indicate to personnel that an order has been placed. Such personnel may then at least partially fulfill the order by physically sending a storage device containing the waveform file (or a digital signal representative thereof) to the client.

A waveform file may comprise instructions for a device to generate a waveform having a shape, magnitude, or frequency based, at least in part, on one or more characteristics of one or more patients, wherein such characteristics may comprise one or more of: body weight, age, sex, heart condition, injury status, injury or health history, and history of treatment using the device, just to name a few examples. The waveform file may further comprise a time code that indicates an expiration time of the treatment waveform.

In one implementation, an order a provider may receive from a client may comprise a renewal order to add time to an expiration time of a waveform file.

In an embodiment, a method performed by a client updating performance characteristics of a device may comprise placing an order to a provider for a waveform file for a waveform to be electrically applied by the device to one or more patients. After receiving (e.g., via the Internet or via transportable memory) a waveform file, a client may provide the waveform file to a device. The device may then be used to electrically apply the waveform to one or more patients. In one implementation, providing a waveform file to a device may comprise storing a received waveform file in a memory device, and connecting the memory device to the device to enable the waveform file in the memory device to upload into the device.

As mentioned above, a waveform file for a waveform may comprise an expirable waveform file that expires after a predetermined time that the waveform is applied to one or more patients. Accordingly, in some cases, an order placed by a client may comprise a renewal order to add time to an expiration time of such an expirable waveform file.

A waveform may have properties that satisfy particular criteria. (Herein, “criteria” is used interchangeably with “criterion”, so that “criteria” means a single criterion or multiple criteria. Also, unless the context of a sentence indicates otherwise, “criteria” may be used interchangeably with the term “rules” or “rule”, which is intended to comprise plural or singular.) For example, energy per pulse of a waveform applied to a patient may be below a particular upper limit set forth by a regulatory agency. Modifying such a waveform, however, may give rise to altered properties that no longer satisfy particular criteria. For example, increasing a pulse width of a waveform may increase energy per pulse beyond a particular upper limit set forth by a regulatory agency.

Criteria or rules set forth by an agency or other entity need not be “hard-wired” into electronics or software code of a device. For example, a regulatory agency may set forth a rule that a waveform applied to a patient is not to exceed a peak voltage of 35.0 volts. In one implementation, a waveform or waveform file may be approved by a regulatory entity. A device may be constructed with electronic components or software so that the device does not have a capability to exceed a peak voltage of 35.0 volts. However, in some embodiments, a device may be “over-built” in a sense that the device may be able to exceed limitations or operate beyond ranges set forth by an agency, group, or individual (e.g., a device may be built to exceed a peak voltage of 35.0 volts). Output capability, instead, may be reigned in or limited by values of a waveform file maintained in a memory. Such values may be updated from time to time or periodically, such as in response, for example, to a regulatory agency issuing new criteria. Such values may comprise data in a look-up table, for example. Values may comprise any of a number of parameters that describe a waveform, such as voltage, current, energy per pulse, power, frequency, rate of change of voltage, waveshape (e.g., ramp, sinusoid, square, or arbitrary shape), and so on. Thus, returning to the example, above, a device may be constructed to reach peak voltages of 100 volts, and include memory storing a variable that specifies output of the device is to not exceed a peak voltage of 35.0 volts. Such a device may provide a number of advantageous, including a device that may easily be adaptable to changing regulations, conditions, or preferences, for example.

In some embodiments, a device may be constructed so as to generate an electronic waveform having an arbitrary shape. Parameters of such a waveform may depend, at least in part, on its waveshape. For example, if a waveform is graphed as voltage versus time, energy per pulse of the waveform may be proportional to the area under the graph. Accordingly, energy per pulse may change as a shape of the pulse changes.

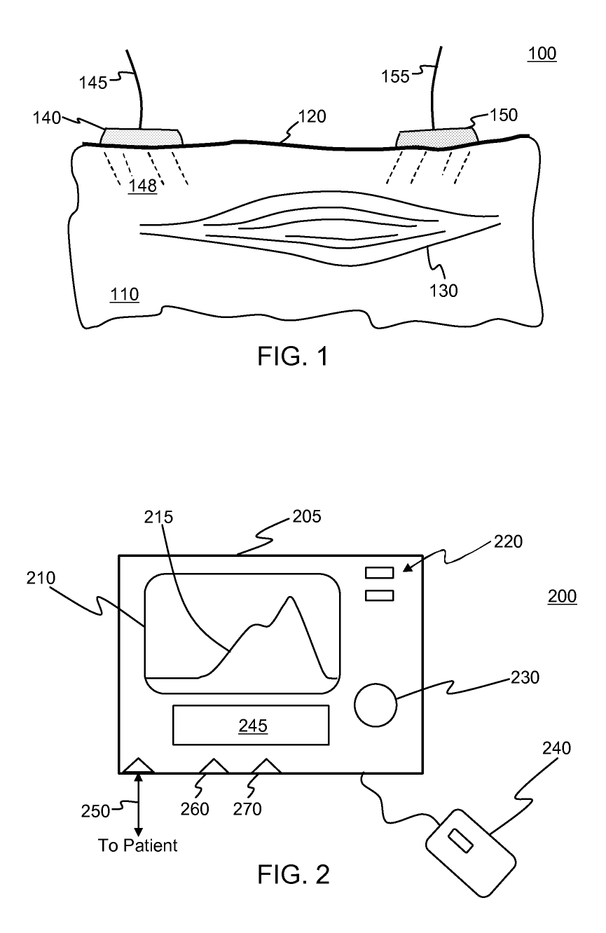

FIG. 1 is a cross-sectional schematic diagram illustrating electrodes 140 and 150 for applying or distributing one or more electrical signals to a portion 110 of a patient, according to an embodiment 100. In examples provided herein, electrodes may comprise pads to be applied to a patient's skin. However, electrodes may comprise any of a number of conduction vehicles to apply an electrical waveform to a patient, such as needles, wire loops, clamps, clips, and so on. Electrodes may further comprise articles of clothing. For example, a shirt, undergarments, gloves, hat, or portions thereof, may be at least partially conductive so as to apply an electrical waveform to a patient wearing such items. Such garments may fit onto a patient in a skin-tight fashion, for example.

Portion 110 may comprise a volume of body mass including skin 120 and muscle 130. For sake of clarity, portion 110 may include other biological elements or material that which are not shown. For example, such biological elements or material may comprise DNA, normal or cancer cells, fascia, bone, ligaments, organs, plasma, blood vessels, arteries, and so on. Leads 145 and 155 may carry electrical signals to/from electrodes 140 and 150, respectively. A general flow of electrical signals is schematically indicated by symbol 148. Electrodes 140 and 150 may comprise a self-adhesive, metal foil, or conductive rubber (e.g., carbon-impregnated silicone rubber) electrode. In some implementations, a coupling medium may be used to provide a conductive bridge between the electrode and the skin, such as by filling in voids or gaps, or by increasing conductivity of skin or electrode surfaces. A coupling medium may be an integral part of self-adhesive electrodes, for example. With conductive rubber electrodes an adhesive gel pad may be used. A coupling gel-pad, which may be solid but soft and flexible, may be both electrically conductive and adhesive. Electrodes may also be strapped onto skin, with or without a coupling medium. A coupling medium for metal foil electrodes may comprise an electrode gel or a wetted pad of lint, cotton gauze, or some form of sponge material that absorbs and retains water, for example. Metal electrodes using spread-able gel or wetted pads may be held in contact to skin by straps or bandages.

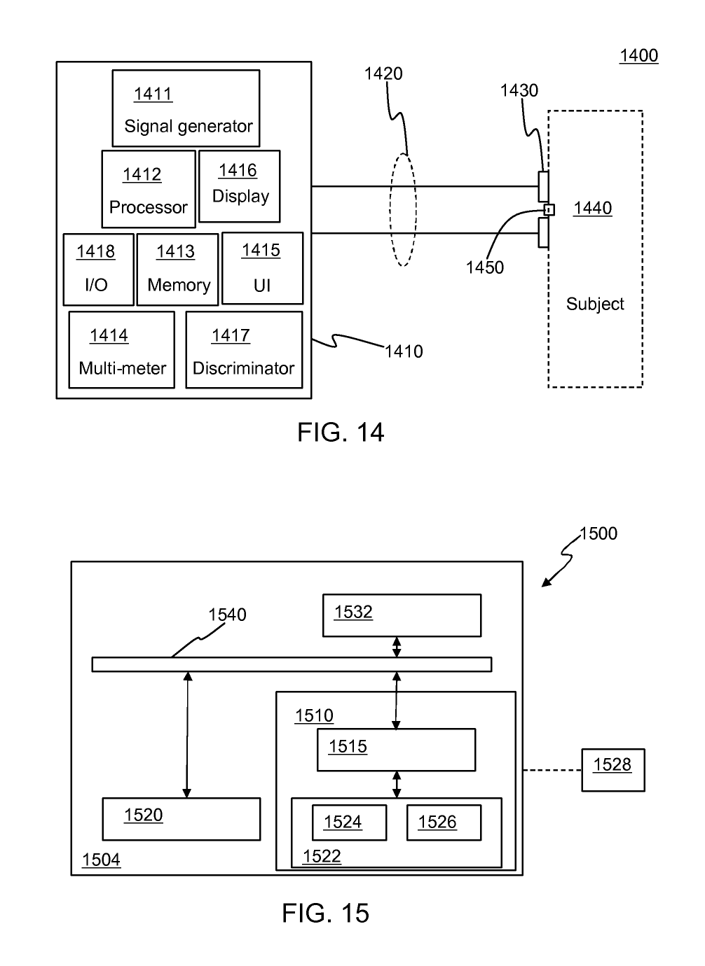

FIG. 2 is a schematic diagram illustrating a device 205, which may comprise a medical or therapeutic device, according to an embodiment 200. Device 205 may apply a waveform to a patient (e.g., 1440 in FIG. 14) via port 250, according to an embodiment. Device 205 may generate a waveform to be applied to a patient via electrodes, for example, such as 140 and 150. The waveform may be generated based, at least in part, on a digital signal downloaded from the Internet via Internet connection 270 or downloaded from another source via USB port 260, for example. Such a digital signal may comprise an electronic file (e.g., 400) that includes computer-readable code representative of one or more waveforms, for example. A waveform may attain any of a number of shapes. For example, a waveform may comprise a sinusoid, a square wave, a sawtooth wave, a low-duty-cycle pulse, microcurrent wave, or an arbitrarily-shaped wave. A waveform may comprise one waveshape (or other parameters) for one time span, another waveshape (or other parameters) for a subsequent time span, and so on. Variables of waveforms may include time between pulses, pulse duration, duty cycle, shape of pulses, frequency modulations, amplitude modulations, pulse width modulations, ramping, peak on-times, surging, decay rates, and so on. In the example shown, waveform 215 may comprise a pulse including two peaks. Such waveforms are merely examples, and claimed subject matter is not limited to any particularly-shaped wave or signal. Device 205 may include a screen 210, which may comprise a touchscreen, for example. Device 205 may include a number of switches 220, knobs 230, or keyboard 245 to allow a user to manipulate the device, input patient information, adjust parameters of a waveform, and so on, for example. A waveform file executable by device 205 may include instructions regarding sensitivity or functionality of any of switches 220, knobs 230, or keyboard 245, for example. Such instructions may also be executable by device 205 to operate screen 210 with particular features or have particular functionalities.

In one implementation, a graphical representation of waveform 215 may be changed or adjusted by a user via touchscreen 210. In another implementation, a graphical representation of waveform 215 may be changed by a user via mouse 240. In yet another implementation, waveform 215 may be changed in response to feedback or other signal provided at port 250, as explained below. Of course, such details of device 205 are merely examples, and claimed subject matter is not so limited.

Though device 205 of embodiment 200 is shown in FIG. 2 to have various features or components, a device may comprise any of a number of configurations. For example, in one embodiment, a device may comprise an amplifier that receives (e.g., wired or wirelessly) and amplifies electronic signals representative of a waveform. Such a device need not include a processor, for example. In such a case, in one implementation, a processor to execute code of a waveform file may reside in an electronic device external to a device. Such electronic devices may comprise a smartphone, mobile phone, touch pad, laptop, and so on.

In one embodiment, a device (e.g., 1600, described below) may comprise a smartphone, mobile phone, touch pad, laptop, or other portable (or non-portable) electronic device. Such a device may comprise an input port to receive a waveform file for a waveform to be electrically applied to one or more patients via an output port. For example, an input port may comprise a wireless receiver (e.g., Bluetooth) or a mini- or micro-USB port or other wired connection. An output port may comprise a wireless transmitter, mini- or micro-USB port or other wired connection, or a headphone jack (e.g., monaural or stereo). The device may further comprise electronics configured to generate a waveform having a shape, magnitude, or frequency based, at least in part, on a waveform file. The electronics may further provide the waveform to the output port. In one implementation, such a waveform may comprise a microcurrent waveform. For example, output capability of a headphone jack of a smartphone may be sufficient to apply hundreds of micro-amps to a patient having an impedance of tens of kilo-ohms. In another implementation, an external amplifier may be used with a smartphone (or other portable electronic device), for example, to amplify relatively small voltage or current amplitudes output by the smartphone to higher values sufficient for application to a patient.

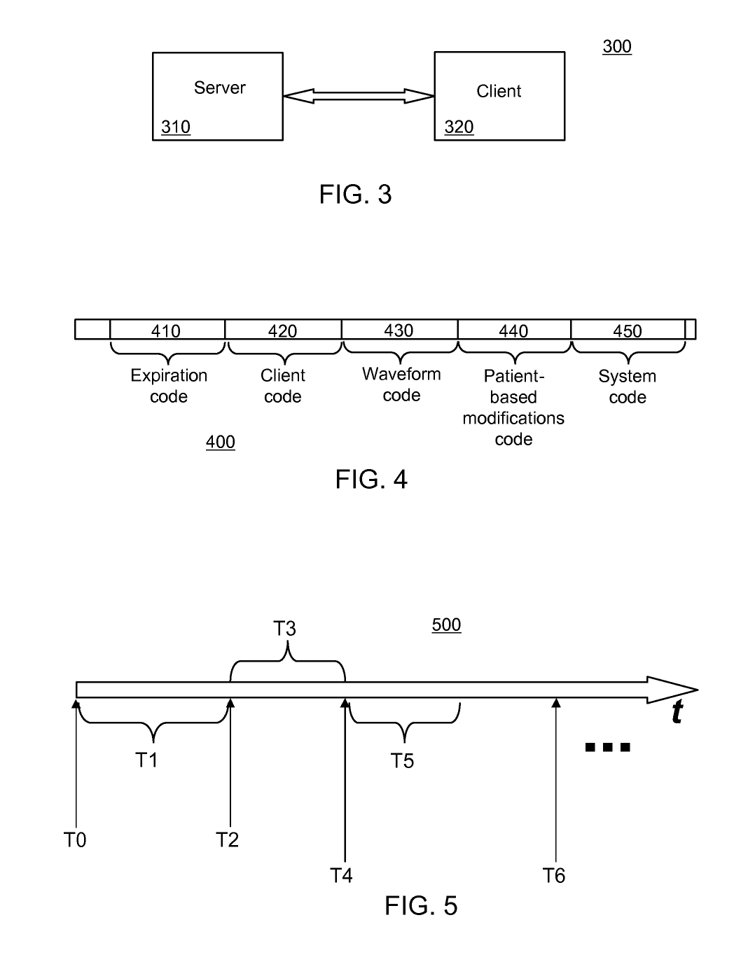

FIG. 3 is a schematic block diagram illustrating a system 300 for exchanging waveform files between a server 310 and one or more clients 320, according to an embodiment. A client 320 may include a device, such as 205 for example.

FIG. 4 is a schematic diagram illustrating a waveform file 400 comprising a packet of data or other code format, according to an embodiment. Waveform file 400 may comprise a digital signal comprising at least a portion of an electronic file that includes computer-readable code representative of one or more waveform files.

Waveform file 400 may be electronically transmitted from server 310 to one or more clients 320 via the Internet or stored and moved in a transportable memory device, for example. In one implementation, waveform file 400 may be transmitted from a smartphone to a device via a wired (e.g., USB) or wireless (e.g., Bluetooth) connection.

In detail, waveform file 400 may comprise code that may or may not be partitioned into one or more functional portions. For example, waveform file 400 may include an expiration code 410, a client code 420, a waveform code 430, a patient-based modifications code 440, or a system code 450. Of course, such code portions may be arranged in any order, or may be partitioned in any fashion, and claimed subject matter is not so limited.

Expiration code 410 may comprise computer-readable code that indicates expiration data pertaining to expiration of the waveform file 400. An expired waveform file may no longer be operational, for example. In one implementation, an expired waveform file on a particular device may no longer be operational by the particular device. For example, expiration data may describe a time or date that a waveform file expires (e.g., waveform file to expire at 6:30 PM, September 29). In another example, expiration data may describe a time span after which a waveform file expires (e.g., a waveform file to expire 30 days from day of receipt by client). In yet another example, expiration data may describe a usage time after which a waveform file expires (e.g., waveform file to expire after 100 hours applied to one or more patients). In still another example, expiration data may indicate that a waveform file never expires. For example, a particular waveform file may expire for some clients and never expire for other clients. Such different expiration conditions may be based, at least in part, on price paid by clients, class or group of clients, and so on. For example, for a particular waveform, a client may pay triple a particular price for a waveform file that never expires compared to a waveform file that expires in 30 days. In another example, for a particular waveform, universities or research institutions may receive a waveform file that never expires, whereas commercial, for profit groups may receive a waveform file that expires after 100 hours of use unless a new time period is paid for. Of course, claimed subject matter is not limited to such numerical examples.

Client code 420 may comprise computer-readable code that identifies a particular client or group of clients. A client may comprise and individual (e.g., patient, healthcare practitioner, clinic, and so on) or an entity, such as a club, group, or class of clinics, medical associations, patients, and so on. Client code 420 may be used as an address during electronic transmission of waveform file 400. For example, if waveform file 400 is broadcast over a computer network, a particular client associated with client code 420 may retrieve waveform file 400 upon or after recognizing the client code. In this example, other clients associated with a different client code may ignore waveform file 400 or not privileged to retrieve the waveform file. Client code 420 may be used to restrict operation of a waveform file to all but one or a group of devices. For example, a waveform file may be intended to be non-transferable among devices. In such a case, a device may be configured to disallow generation of a waveform of the waveform file if client code 420 identifies a different device. Thus, in some embodiments, individual devices (e.g., 205) may comprise unique identification, which may be compared to client code 420, for example. Identification may comprise hardware or software configured during or after manufacture of the device, though claimed subject matter is not so limited.

Waveform code 430 may comprise computer-readable code that describes a waveform by specifying one or more parameters of the waveform. For example, code for a waveform may include enough information to allow a device to generate the waveform. A waveform may be described by any of a number of techniques. For example, one or more mathematical equations may describe, at least in part, a waveform (e.g., amplitude multiplied by a cosine function having a particular frequency, or a series of sinusoidal functions respectively having varying frequencies or amplitudes). In another example, a table of values may describe a waveform. Such a table may include wave amplitude as a function of elapsed time of a cycle of the waveform. Waveform code 430 may describe combinations of waveforms that may, for example, be described as a first waveform for a first time span, a second waveform for a second time span, a third waveform for a third time span, and so on. For example, waveform code 430 may be executed by a device to generate a wave comprising: an exponentially-rising/decaying pulse having a frequency of 500 hertz (Hz) for 2.0 seconds, the exponentially-rising/decaying pulse having a frequency of 800 hertz (Hz) for 10.0 seconds, the exponentially-rising/decaying pulse returning to a frequency of 500 hertz (Hz) for 2.0 seconds, and repeat.

Waveform code 430 may also provide instructions to a device regarding which, if any, parameters of a waveform may be adjusted or modified, and by how much, by a user of the device. In other words, a waveform file for a waveform may include permissions or limitations regarding which, if any, parameters of the waveform may be changed, adjusted, or manipulated by a user operating a device that generates the waveform. A device, for example, may comprise an adjustment control (e.g., knob, lever, touchscreen, etc.) to adjust “volume” or amplitude of waveforms that are generated and output by the device. An extent to which an adjustment control of a device may adjust amplitude of a particular waveform may depend, at least in part, on instructions of code 430 (or any other portion of packet 400) for the particular waveform. For example, peak voltage of a first waveform may be adjustable from zero to 50.0 volts, whereas peak voltage of a second waveform may be adjustable from zero to 100.0 volts. Continuing this example, a peak voltage of the second waveform may be allowed to be adjusted to a value higher than that of the first waveform if a pulse width of the second waveform is narrower than that of the first waveform (all other things being equal): A maximum allowable energy per pulse may be the same for both waveforms. Other parameters of waveforms that may be adjustable (e.g., by permission included in code 430) include current amplitude, pulse width, frequency, frequencies, frequency ranges, time spans (e.g., of one portion of the waveform versus another portion), and so on.

A device, in another example, may comprise an adjustment control (e.g., knob, lever, touchscreen, etc.) to adjust frequency or frequencies of waveforms that are generated and output by the device. An extent to which an adjustment control of a device may adjust frequency or frequencies of a particular waveform may depend, at least in part, on instructions of code 430 (or any other portion of packet 400) for the particular waveform. For example, frequency of one component of a waveform may be adjustable from 20 to 500 hertz, whereas frequency of another component of the waveform may be adjustable from 5000 to 20000 hertz.

In an implementation, code 430 regarding a waveform may comprise instructions that constrain the waveform to a single (e.g., non-adjustable) frequency or amplitude.

Patient-based modifications code 440 may comprise computer-readable code that describes instructions or waveform characteristics for one or more particular patients or class of patients. For example, code 440 may indicate that patients with a particular heart condition are not to receive a waveform having a frequency in a particular range. Accordingly, operational parameters of a device may be modified upon or after the device reads code 440. Continuing with the example, a device operating with code 440 (of this particular example) may not generate or output to a patient a waveform having a frequency in a particular range if the patient has a particular heart condition. Information regarding a patient may a priori be entered into a device via touchscreen 210, keyboard 245, mouse 240, memory upload, and so on, for example.

In one implementation, a device may generate a waveform having a shape, magnitude, or frequency based, at least in part, on patient-based modifications code 440 and on patient information regarding one or more patients, wherein patient information may comprise one or more of: body weight, age, sex, heart condition, injury status, injury/health history, and history of treatment using the device. Thus, patient-based modifications code 440 may be used to instruct a device to operate particular ways or to modify a waveform for particular patients, for example. Information regarding one or more patients may be stored in a device or may be stored in an external memory (memory storage that is not integral to the structure of the device, such as memory disk, memory stick, hard-drive of a laptop computer, and so on). Information regarding patients may comprise any of a variety of data structures, such as data tables, data groupings partitioned for individual patients, and so on. A processor may generate a waveform based, at least in part, on information regarding one or more patients and code 440.

In one implementation, code to instruct a (processor of a) device on how to modify a waveform for particular patients may comprise code representing mathematical formulas (e.g., a sine function having an amplitude proportional to patient weight) that the device may calculate to determine any of a number of waveform parameters. In another implementation, code to instruct a device on how to modify a waveform for particular patients may comprise code representing one or tables of values of any of a number of waveform parameters.

System code 450 may comprise computer-readable code that, among other things, may be used to adapt code of waveform file 400 to particular types of devices. For example, particular code may not be readable by different types of devices (e.g., different models, different manufacturing dates, different operational code, different hardware, and so on). To allow for such device variability, system code 450 may comprise drivers or routines that allow various devices to read waveform file 400.

In another embodiment, code may be readable by a device to unlock one or more waveform files stored in the device. Such code may be downloaded onto a device or entered by a user via a user interface of a device. For example, a particular device may be loaded with a plurality of waveform files at the time of manufacture or some time after. A client purchasing the device may or may not have a privilege to operate one or more of these waveform files: the device may be configured to not generate a waveform without first acquiring a particular code to unlock a waveform file. Such a code (or codes) may be acquired upon purchase, for example, via the Internet (e.g., email), a transportable memory device, and so on. In one implementation, a user may enter a code, via keypad for example, into a device. In another implementation, a code may be downloaded into a device automatically via the Internet or wireless transmission (e.g., from a wireless access point), without user involvement. In yet another implementation, a code may expire after some time span or after a usage time of a waveform file for which the code was used. In such a case, the same waveform file may be unlocked again after expiration, but by a code that may be different from the first code.

FIG. 5 is a schematic diagram illustrating a time sequence 500 involving waveform file expiration, according to an example embodiment. At time T0, a client may receive an expirable waveform file onto their device that is to expire at the end of time span T1 or at time T2. Upon or after expiration, for example, a waveform of the waveform file may no longer be generated or output by the device. During time span T1, the client may place an order to extend expiration of the waveform file by an additional time span T3 or to time T4. During time span T5, after the waveform file has expired and the device may no longer produce a waveform of the waveform file, the client may desire to renew the waveform file. A server or waveform file provider may offer the client several expiration options. For example, for one fee the waveform file may be set to expire at time T6, and for another (probably higher) fee the waveform file may be set to never expire.

In other example embodiments, expiration of a waveform file downloaded (e.g., or stored) on a device may be based, at least in part, on elapsed time that a waveform of the waveform is generated or output by the device. For example, a waveform file downloaded to or stored in a device may be set to expire after 20 hours of the device applying the waveform to one or more patients.

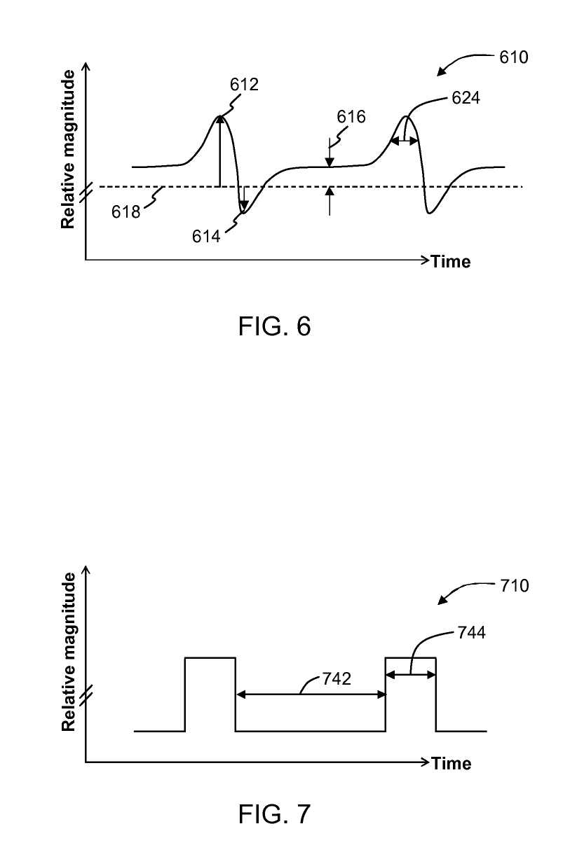

FIGS. 6 and 7 show example waveforms plotted as magnitude of voltage or current versus time, according to embodiments. For example, a waveform applied to a patient via electrodes may comprise any such wave or variation thereof. Of course, there are an endless variety of waveforms having different shapes or characteristics, and FIGS. 6 and 7 show merely a small number of possibilities. Here, the figures are useful for helping to explain meanings of some terms that are used to describe waveforms characteristics.

In particular, FIG. 6 shows a wave 610 that includes a positive-going peak magnitude 612 (e.g., curve is concave downward), a negative-going peak magnitude 614 (e.g., curve is concave upward), and an offset 616 from a reference level 618, which may be zero volts or ground, for example. Wave 610 also includes a width 624 (e.g., pulse width), which may be described as full width at half max (FWHM). In FIG. 7, wave 710 comprises a square wave having a pulse width 744 and duty cycle that may be described by time 742 between pulses. Of course, any wave may be described by any parameters introduces above, and claimed subject matter is not so limited.

Another example of a waveform is described in U.S. Pat. No. 5,109,835 to Thomas (1992). FIGS. 2A-2C in Thomas show examples of a periodic-exponential waveform that may be used for electrotherapeutic treatment. Such a waveform may comprise an interferential current that arises from “beating” of two or more waveforms having different frequencies.

Yet another example of a waveform comprises a microcurrent waveform, which may be used in various therapies. A microcurrent waveform may have peak amplitudes in the order of hundreds of micro-amps or less, for example. On the other hand, other waveforms (non-microcurrent) described herein may have currents in the order of tens of milli-amps or higher than 100 milli-amps.

Still another example of a waveform is described in U.S. Pat. No. 4,683,873 to Cadossi et al. (1987). FIG. 2 in Cadossi et al. show an example of an electromagnetically-induced waveform that may be used for electrotherapeutic treatment. Such a waveform may comprise a microcurrent waveform (e.g., having a current density in the range of 2-30 micro-amps per square centimeter).

Other examples of waveforms include continuous sinusoid, rectangular alternating current (AC), burst modulated, sinusoidal AC, sinusoidal amplitude modulated, monophasic or biphasic pulsed current (symmetrical or asymmetrical), ramping pulse current, Faradic current, and Russian current, just to name a few examples. Of course, waveforms and claimed subject matter are not limited to such examples. For example, some useful and effective waveform shapes to treat some conditions of a patient may not be realized yet, or may not be approved yet as safe by a government agency. But as some waveform shapes become available, a waveform file may be developed to provide instructions to a device to generate the waveform shapes.

In a particular implementation, intensity values of waveforms may change by any amount or fashion. Though, in one embodiment, a waveform file of a waveform or a device for applying the waveform may constrain or modify such changes in intensity values (e.g., as explained regarding code 440 or criteria set forth by an entity). Such a change may be desired, or proposed, by a user based, at least in part, on a particular situation at hand. For example, a user may desire to increase a value of one portion of a waveform while decreasing another portion of the waveform because such a change may affect a particular organ of a patient over another. Here, the meaning of “intensity values” may include values of voltage or current of any portion of a waveform, such as a positive peak (e.g., 612), a negative peak (e.g., 614), an offset (e.g., 616) of a wave from a reference (e.g., ground), and so on.

In another particular implementation, frequencies of waveforms may change by any amount or fashion. Though, in one embodiment, a waveform file of a waveform or a device for applying the waveform may constrain or modify such changes in frequency (e.g., as explained regarding code 440 or criteria set forth by an entity). Such a change may be desired, or proposed, by a user based, at least in part, on a particular situation at hand.

In yet another particular implementation, waveforms may be used for pleasure or relaxation. For example, applying waveforms to a patient may have one or more similar effects as any of a number of types of massage therapies or emotional stress reduction. In some cases, a waveform file may include instructions to constrain peak voltages or currents of a waveform to relatively low values so that a patient need not experience anything but a comfortable, pleasurable treatment with the applied waveform.

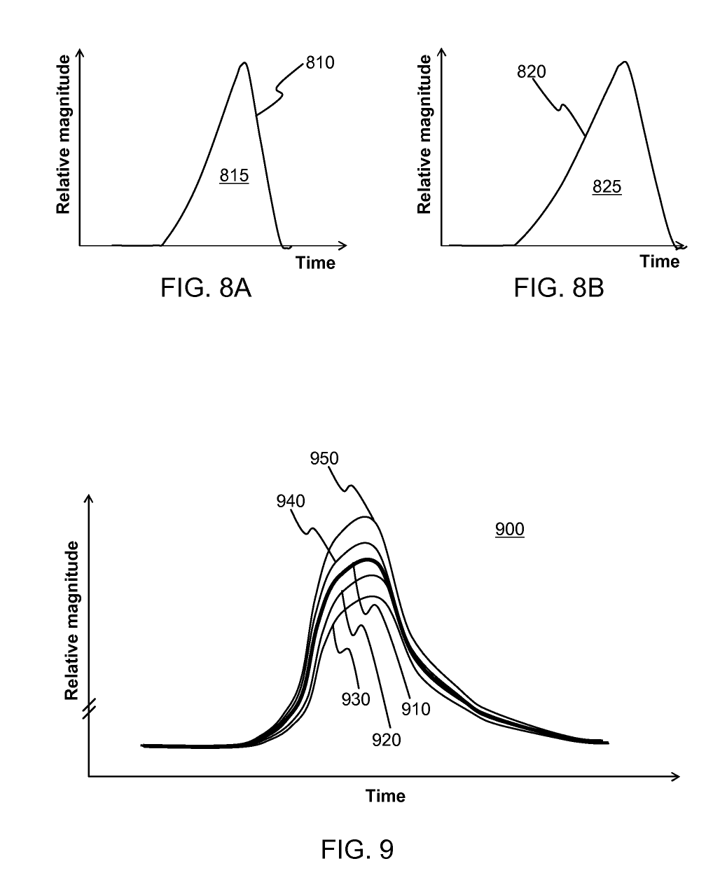

FIGS. 8A and 8B show at least a portion of a first waveform 810 and a second waveform 820, respectively, plotted as magnitude of voltage or current versus time, according to an embodiment. Waveforms 810 and 820 may be similar except that a pulse width of waveform 820 may be greater than that of waveform 810, for example. Waveforms 810 and 820 may both be derived, at least in part, from a waveform file downloaded to a device. Instructions included in the waveform file may specify if or how the pulse width, among other things, of the waveform is to be modified based, at least in part, on characteristics of intended patient(s). For example, such instructions may specify that energy of a pulse applied to a patient weighting below 100 lbs may not exceed 250 milli-joules (mJ). Accordingly, the original waveform having a pulse energy of 300 mJ may be reduced to waveform 810 having a pulse energy of 250 mJ or less (the pulse energy may be lowered by a user operating a device).

For another example, instructions may specify that energy of a pulse applied to a patient weighting over 200 lbs may not exceed 300 mJ. Accordingly, the original waveform having a pulse energy of 300 mJ may remain unchanged to comprise waveform 820 having a pulse energy of 300 mJ or less (the pulse energy may be lowered by a user operating a device). The energy of a pulse may be proportional to the area under the pulse curve, so that pulse energy of waveform 810 may be proportional to area 815, and pulse energy of waveform 820 may be proportional to area 825, for example. Of course, values of other parameters besides pulsewidth may be specified in instructions, such as frequency, voltage, zero-offset, and so on.

FIG. 9 shows at least portions of several waveforms for various applications, plotted as magnitude of voltage or current versus time, according to an embodiment 900. As mentioned above, a device may generate an electronic waveform having a shape, magnitude, or frequency based, at least in part, on patient information regarding one or more patients, wherein patient information may comprise one or more of: body weight, age, sex, heart condition, injury status, injury or health history, and history of treatment using the device. In the example embodiment 900, waveform 910 may comprise an original or default waveform of a waveform file downloaded or stored in a device, such as 205, for example. The device may modify any of a number of parameters of waveform 910 to account for characteristics of a patient to which the waveform is to be applied. As discussed above, a code (e.g., included in waveform file 400) may be representative of instructions on how to modify a waveform. For example, waveform 910 may be modified to waveform 920 for a patient with a particular injury. Or waveform 910 may be modified to waveform 930 for a patient with another particular injury. Or waveform 910 may be modified to waveform 940 for a patient with yet another particular injury and who has had no previous treatments. Or waveform 910 may be modified to waveform 950 for a patient with the same particular injury and who has had several previous treatments. And so on. In these examples, waveform 910 may be modified by changing voltage or current magnitude of a pulse. However, any of a number of other parameters, such as peak or average voltage, peak or average current, energy per pulse, energy per cycle, frequency components, zero-offset, pulse width, slope, decay rate, rise time, and so on, may be changed in view of patient characteristics.

Though a device may modify a waveform in view of patient characteristics and in view of a waveform file, such modification may be constrained to comply with various criteria, such as those set forth by a regulatory agency. Further, a user may adjust magnitude or other parameters of a waveform by operating knobs or other controls of a device. Such adjustments may also be constrained to comply with various criteria, such as those set forth by a regulatory agency. Accordingly, in one implementation, a device or a waveform file may include instructions about how modifications or adjustments may be made to a waveform while maintaining compliance with criteria or rules set forth by a regulatory agency.

In further detail, any of a number of features of a waveform may be evaluated, including any of a combination of peak or average voltage, peak or average current, energy per pulse, energy per cycle, frequency components, zero-offset, pulse width, slope, decay rate, rise time, and so on. Such evaluation may be in view of one or more criteria or rules set forth by any entity, including a government agency (e.g., the Food and Drug Administration (FDA), Federal Communications Commission (FCC), or Federal Aviation Administration (FAA)), or a committee or governing body of a group (a medical group overseeing patient treatment, by a patient (e.g., 1440) to which a waveform may be applied, by a medical practitioner treating a patient, or by a researcher investigating a patient, just to name a few examples. In one particular implementation, one or more criteria or rules may be based, at least in part, on safety or medical history of a patient, just to name a few examples. For example, a rule may set forth a relatively low maximum voltage of a signal for a patient with a preexisting heart condition. Another rule may set forth a relatively high maximum voltage of a signal for a young, healthy patient. One or more criteria or rules may be stored in a memory of a device or included in a data packet of a waveform (e.g., 440).

For example, the International Electrotechnical Commission (IEC) sets forth a rule that pulse energy for pulse durations of less than 0.1 seconds shall not exceed 300 mJ per pulse (e.g., IEC 60601-2-10, section 201.12.4.104, Limitation of Output Parameters).

In another example, a human patient to which waveforms are applied may set forth maximum values of voltage or energy of the waveforms. In an example implementation, an electrical muscle stimulator may apply a waveform transcutaneously to a patient. Though the waveform may benefit the patient (and the patient selects such treatment), the waveform may be uncomfortable, more so for some patients. Comfort level may be proportional to voltage level of an applied waveform, for example. Accordingly, it may be desirable for patients to have an opportunity to select maximum values of voltage or energy, among other things, of waveforms based, at least in part, on the patient's anticipated comfort level, for example.

In some embodiments, a proposal to change magnitude of a waveform may be generated by a knob or other control device that a user may operate, as mentioned above. For example, a user may desire to increase amplitude of a waveform to a particular value. However, such a particular value, in view of other parameters (e.g., any of a combination of peak or average voltage, peak or average current, energy per pulse, energy per cycle, frequency components, zero-offset, pulse width, slope, decay rate, rise time, and so on) may lead to a modified waveform that would violate criteria or rules. For example, a relatively narrow pulse width may allow for a relatively high peak voltage, whereas a relatively wide pulse width may preclude such a relatively high peak voltage. Accordingly, a processor receiving the proposal may predict one or more parameters of the modified waveform, and may compare the one or more parameters to criteria such as corresponding one or more threshold values. The processor may subsequently determine whether to reject or accept the modified waveform based, at least in part, on the comparing of parameters to threshold values or whether the modified waveform complies with the criteria.

In some embodiments, a proposal to modify a waveform may be generated graphically (e.g., by a user via a mouse or touchscreen). In other embodiments, a proposal to modify a waveform may be generated in response to feedback based, at least in part, on the waveform applied to a patient. For example, a waveform may be applied transcutaneously via cables and electrical pads to a patient. A response of a patient to a waveform may give rise to a feedback signal that may be representative of a physical condition of the patient, as described below.

As indicated above, a waveform may be used as a diagnostic tool to measure impedance of biological elements of the patient: A waveform comprising an electrical signal may follow a path depending, at least in part, on electrical and/or chemical properties of internal portions of a patient. For example, electrical conductivity of muscle may be different from that of bone or a particular organ. Moreover, as an example, electrical conductivity of muscle tissue or bone may depend, at least in part, on the health or density of the muscle tissue or bone (or portion thereof). In the case of muscle tissue, for example, measurements of electrical conductivity of muscle tissue may be used to determine muscle loss or gain in patients with Lou Gehrig's Disease, also known as amyotrophic lateral sclerosis, or ALS. This disease may attack motor neurons that control voluntary muscle movement, leading to muscle weakness and atrophy. As ALS spreads, motor neurons may die off, causing muscles to atrophy. Deteriorating muscles may behave differently from healthy ones, resisting electrical current more, for example. Such variations in behavior may be correlated with disease progression and length of survival of a patient. As another example, electrical conductivity of internal portions of a patient may depend, at least in part, on tissue density, presence of cancer cells, and so on.

Biological elements may respond to different waveforms in different ways. For example, a pulse of a waveform may activate an action potential of nerve fibers in muscle tissue if a slope of the pulse is sufficiently steep. On the other hand, if a pulse is not steep enough, then the same nerve fibers may accommodate (e.g., “adjust”) to current flow of the pulse so that no action potential is activated. This illustrates an example where applied waveforms may affect biological elements for which the waveforms are used to diagnose. For another such example, a waveform applied to muscle tissue may increase permeability of the muscle tissue. Accordingly, application of particular waveforms may affect muscle tissue so that resistance of the muscle tissue changes in response to the applied waveforms. Different applied waveforms (e.g., different by frequency, waveshape, voltage level, and so on) may affect particular biological elements differently. Thus, for example, different applied waveforms may give rise to different resistances of a particular biological element, which may give rise to particular feedback signals fed back to the source of the applied waveforms.

A feedback signal may travel in the same cables and electrical pads as that of the waveform or may travel in a second set of cables and electrical pads. In response, at least in part, to evaluating a feedback signal, a processor may execute code that determines whether or not the waveform is to be modified, and if so, how it may be modified, such as in view of rules or criteria set forth. For example, a processor may determine that a feedback signal based, at least in part, on a waveform applied to a patient indicates that a voltage of the waveform should be increased by a particular amount to have a desired effect on the patient. The processor may consequently generate a modified waveform having an increased voltage. However, the processor may further determine whether such a modified waveform would violate any rules or criteria. For example, an increased voltage of a modified waveform may not violate any criteria, but an associated increase in power may violate criteria. A processor may evaluate a number of parameters to arrive at a modified waveform that finally satisfies a feedback signal and applicable criteria, or may place uneven weight on the feedback signal, the criteria, and/or signal parameters to reach a compromise. Other changes to a waveform, besides voltage, may involve changing its shape, frequencies, magnitude, power, and so on. Of course, such details of processes involving feedback are merely examples, and claimed subject matter is not so limited.

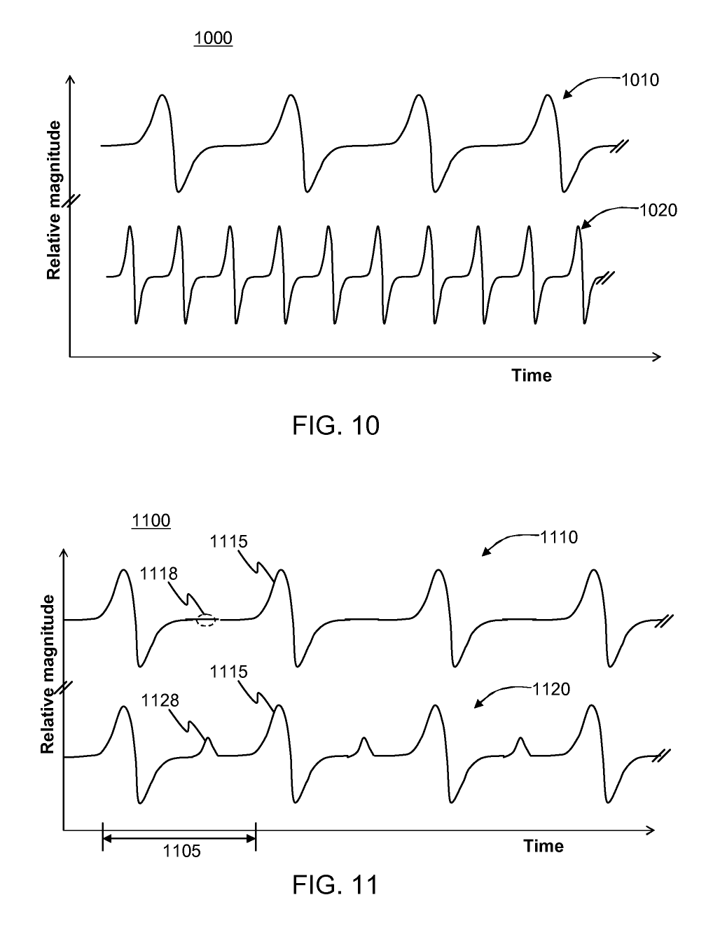

FIGS. 10 and 11 show example waveforms plotted as magnitude of voltage or current versus time, according to embodiments 1000 and 1100. First waveform 1010 and second waveform 1020 have different frequencies. Second waveform 1020 may be derived from first waveform 1010 in view of patient characteristics, for example.

FIG. 11 shows a first waveform 1110 and a second waveform 1120 plotted as magnitude of voltage or current versus time, according to embodiments 1100. Second waveform 1120 may include an inter-pulse peak 1128 between pulses 1115. Waveform 1010 may have one particular distribution of Fourier frequencies, and waveform 1120 may have another particular distribution of Fourier frequencies. For example, by adding the feature 1128, a new Fourier distribution of frequencies may be created. In the example of embodiment 1100, a Fourier distribution of frequencies of waveform 1120 may include frequency components that are about double that of waveform 1010, since about double the number of pulses per cycle 1105 may arise due to peak 1128.

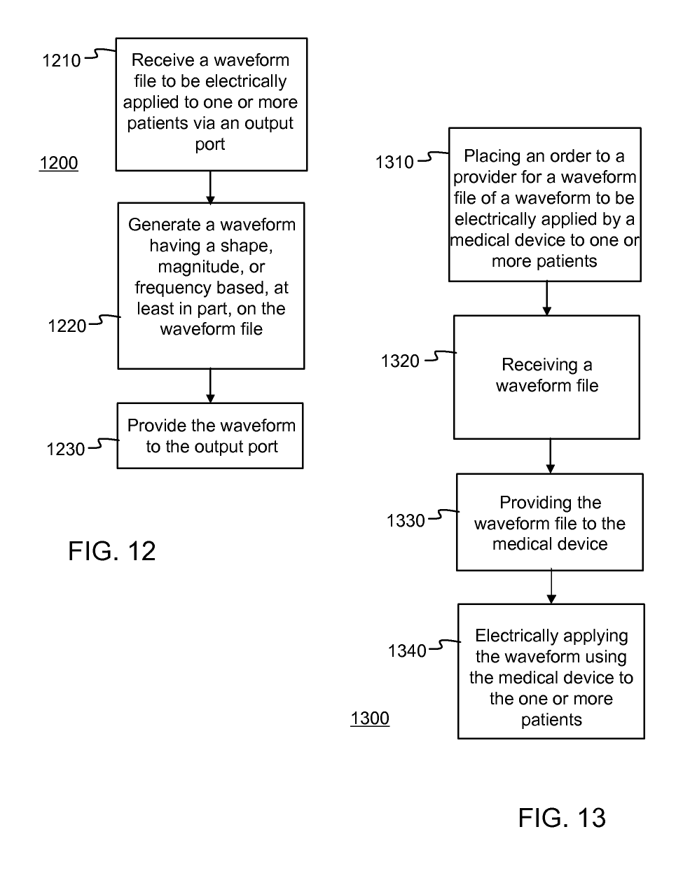

FIG. 12 is a flow diagram of a process 1200 for generating a waveform, according to an embodiment. For example, a device such as 205 may perform process 1200 to electrically apply a waveform to a patient. At block 1210, a device may comprise an input port to receive a waveform file. An input port may comprise a USB port or an Internet port, for example. At block 1220, the device may further comprise electronics to generate a waveform having a shape, magnitude, or frequency based, at least in part, on the waveform file, and to provide the waveform to an output port at block 1230. Of course, such details of process 1200 are merely examples, and claimed subject matter is not so limited.

FIG. 13 is a flow diagram of a process 1300 for ordering a waveform file, according to an embodiment. For example, process 1300 may be performed by a client updating performance characteristics of a device. At block 1310, a client may place an order to a provider for a waveform file of a waveform to be electrically applied by the device to one or more patients. At block 1320, the client may receive a waveform file. At block 1330, after receiving (e.g., via the Internet or via transportable memory) a waveform file, a client may provide the waveform file to the device. In some embodiments, process 1300 may combine blocks 1320 and 1330, such as, for example, if a device receives a waveform file wirelessly from an access point or via the Internet (wired or wirelessly). At block 1340, the device may then electrically apply the waveform of the waveform file to one or more patients. Of course, such details of process 1200 are merely examples, and claimed subject matter is not so limited.

FIG. 14 is a schematic block diagram illustrating a system 1400 for performing a process, such as 1200 or 1300, for example, according to an embodiment. For example, system 1400 may comprise a device 1410, cables 1420, and electrodes 1430. Device 1410 may be similar to device 205 described above, for example. Device 1410 may generate one or more signals that may be applied to a subject or patient 1440 via electrodes 1430. Device 1410 may include a signal generator 1411 to generate waveforms having any of a number of parameters, such as waveshape, magnitude, frequency, offset (e.g., from zero volts), and so on. Signal generator 1411 may generate more than one waveform at a time, or may repeatedly and alternately generate a first waveform and a second waveform. Signal generator 1411 may operate under instructions from a processor 1412, which may execute code of a waveform file to instruct signal generator 1411 to generate a waveform, for example. Processor 1412 may be used to calculate or determine resistance to a waveform provided to electrodes 1430, which may be electrically connected to patient 1440. Processor 1412 may also evaluate feedback provided by cables 1420 to determine any of a number of parameters. In another implementation, processor 1412 may also evaluate output of detectors 1450 provided via cables 1420, other conductors, or wireless transmission (e.g., from detector 1450 to device 1410). Such detectors may measure one or more parameters representative of a physical condition of patient 1440. For example, such detectors may comprise a blood pressure monitor, blood oxygen level monitor, and so on. Processor 1412 may perform evaluations, calculations, or determinations using parameters measured by multi-meter 1414, for example. Such parameters may include voltage, current, phase shift, and so on.

A discriminator 1417 may decompose or separate a composite (e.g., non-sinusoidal) waveform into two or more individual signals. In one implementation, a composite voltage waveform may include a superposition of any number of individual voltage signals. Current of the composite voltage waveform flowing through patient 1440 may be decomposed by discriminator 1417 so that the current is separated into a number of individual current signals, which may be measured by multi-meter 1414, for example. In one implementation, discriminator 1417 may comprise one or more frequency filters (e.g., low-pass, high-pass, or notch filters, and so on) to perform such signal separation. In another implementation, discriminator 1417 may comprise one or more amplitude filters (e.g., involving resistor networks, diodes, etc.) to perform such signal separation. In yet another implementation, discriminator 1417 may comprise one or more waveshape filters to perform such signal separation. In any case, a composite waveform provided to discriminator 1417 (e.g., by cables 1420) may comprise a digital signal. Here, an analog to digital converter (not shown) may be used to convert an analog composite waveform flowing through patient 1440 to a digital composite waveform. Software executed by processor 1412 may be used to identify or distinguish one waveform of one signal from another waveform of another signal in a digital composite signal. With information from such a processor, discriminator 1417 may separate the separate waveforms and multi-meter 1414 may then measure current or voltage of the separated waveforms.

Device 1410 may further include memory 1413 to store values of parameters measured by multi-meter 1414, or generated by processor 1412 or discriminator 1417, for example. Memory 1413 may also maintain data representative of criteria, rules, or regulations set forth by an agency, group, and so on. Memory 1413 may also maintain information regarding one or more patients, waveform files, or values produced by detectors 1450, just to name a few examples. Data may comprise tables of values of ranges, maxima, minima, averages, etc. for any of a number of parameters of a waveform, such as voltage, current, energy, power, rate of change, and so on. A user interface 1415 may include a keypad, mouse, or touchscreen by which a user may provide operational instructions to device 1410. User interface may be used to enter information into device 1410, such information regarding one or more patients or code to unlock resident waveform files, for example. A display 1416 may display any information to a user, including a graphical representation of a waveform provided over cables 1420, or a proposed waveform. Display 1416 may comprise a portion of user interface 1415, and may comprise a touchscreen, touchpad, and so on. Graphical data in display 1416 may be read by processor 1412 in a process of transferring a graphical representation of a waveform from display 1416 to digital values stored in memory 1413. Display 1416 may display a graphical representation of a signal that is present on cables 1420 or may display a graphical representation of a virtual signal that is merely proposed so as to not actually be present on cables 1420. Input/Output 1418 may comprise an Internet port, a USB port, or a port to receive an external memory device, such as a disk, flash memory, and so on, just to name a few examples. Of course, such details of system 1400 are merely examples, and claimed subject matter is not so limited.

FIG. 15 is a schematic diagram illustrating an embodiment of a computing system 1500, for example. Some portions of system 1500 may overlap with some portions of system 1400. System 1500 may be used to perform processes 1200 or 1300, for example. A computing device may comprise one or more processors, for example, to execute an application or other code. A computing device 1504 may be representative of any device, appliance, or machine that may be used to manage memory module 1510. Memory module 1510 may include a memory controller 1515 and a memory 1522. By way of example but not limitation, computing device 1504 may include: one or more computing devices or platforms, such as, e.g., a desktop computer, a laptop computer, a workstation, a server device, or the like; one or more personal computing or communication devices or appliances, such as, e.g., a personal digital assistant, mobile communication device, or the like; a computing system or associated service provider capability, such as, e.g., a database or information storage service provider or system; or any combination thereof.

It is recognized that all or part of the various devices shown in system 1500, and the processes and methods as further described herein, may be implemented using or otherwise including at least one of hardware, firmware, or software, other than software by itself. Thus, by way of example, but not limitation, computing device 1504 may include at least one processing unit 1520 that is operatively coupled to memory 1522 through a bus 1540 and a host or memory controller 1515. Processing unit 1520 is representative of one or more devices capable of performing at least a portion of a computing procedure or process, such as processes 1200 or 1300, for example. By way of example, but not limitation, processing unit 1520 may include one or more processors, microprocessors, controllers, application specific integrated circuits, digital signal processors, programmable logic devices, field programmable gate arrays, and the like, or any combination thereof. Processing unit 1520 may include an operating system to be executed that is capable of communication with memory controller 1515.

In one implementation, an apparatus may comprise an input port (e.g., 1532) to receive a waveform file of a waveform to be electrically applied to one or more patients via an output port (e.g., 1532); and electronics configured to: generate a waveform having a shape, magnitude, or frequency based, at least in part, on the waveform file; and to provide the waveform to the output port. Such electronics may comprise processing unit 1520 or other electronic components, for example.

An operating system may, for example, generate commands to be sent to memory controller 1515 over or via bus 1540. Commands may comprise read or write commands, for example. In response to a write command, for example, memory controller 1515 may perform process 1500 described above, to program memory and to change parity states.

Memory 1522 is representative of any information storage mechanism. Memory may store rules or criteria, signals applied to a patient, output from detectors measuring parameters of a patient, an so on, as explained above. Memory 1522 may include, for example, a primary memory 1524 or a secondary memory 1526. Primary memory 1524 may include, for example, a random access memory, read only memory, etc. While illustrated in this example as being separate from processing unit 1520, it should be understood that all or part of primary memory 1524 may be provided within or otherwise co-located or coupled with processing unit 1520. In one implementation, memory 1522 may be incorporated in an integrated circuit, for example, which may comprise a port to receive error syndromes or other ECC information from processing unit 1520.

Secondary memory 1526 may include, for example, the same or similar type of memory as primary memory or one or more other types of information storage devices or systems, such as a disk drive, an optical disc drive, a tape drive, a solid state memory drive, etc. In certain implementations, secondary memory 1526 may be operatively receptive of, or otherwise capable of being operatively coupled to a computer-readable medium 1528. Computer-readable medium 1528 may include, for example, any medium that is able to store, carry, or make accessible readable, writable, or rewritable information, code, or instructions for one or more of device in system 1500. Computing device 1504 may include, for example, an input/output device or unit 1532.

Input/output unit or device 1532 is representative of one or more devices or features that may be capable of accepting or otherwise receiving signal inputs from a human or a machine, or one or more devices or features that may be capable of delivering or otherwise providing signal outputs to be received by a human or a machine. By way of example but not limitation, input/output device 1532 may include a display, speaker, keyboard, mouse, trackball, touchscreen, etc.

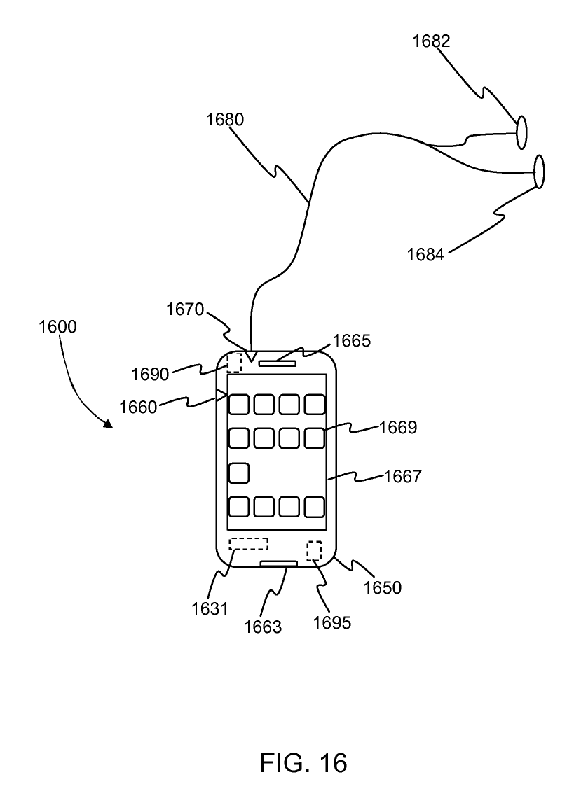

FIG. 16 is a schematic diagram of a device 1600, according to an embodiment. For example, device 1600 may comprise a smartphone, mobile phone, touch pad, laptop, or other portable (or non-portable) electronic device 1650. Herein, a “smartphone” means a portable electronic device comprising a processor, memory, phone, or other functional components (e.g., camera, and so on). In the example embodiments described below, electronic device 1650 is considered to comprise a smartphone for illustrative purposes, but claimed subject matter is not so limited. Smartphone 1650 may comprise speaker 1665, touchscreen 1667, softkeys or adjustment sliders 1669 displayed in touchscreen 1667, or a connector (e.g., for battery charging or other functions) 1663. Though details of a smartphone are given, device 1600 may comprise another type of electronic device, and claimed subject matter is not limited in this respect. Device 1600 may comprise an input port 1660 to receive a waveform file for a waveform to be electrically applied to one or more patients via an output port. For example, an input port may comprise a wireless receiver (e.g., Bluetooth) or a mini- or micro-USB port or other wired connection. In one implementation, device 1600 may wirelessly receive waveform files via a receiver/transmitter 1690 and stores files in memory 1695, for example.

An output port 1670 may comprise a wireless transmitter, mini- or micro-USB port or other wired connection, or a headphone jack (e.g., monaural or stereo). The device may further comprise electronics 1631 configured to generate a waveform having a shape, magnitude, or frequency based, at least in part, on a waveform file. For example, electronics 1631 may comprise a processor configured to execute a waveform file. The electronics may further provide the waveform to the output port. A multi-conductor (e.g., stereo) cable 1680 may plug into output port 1670 and terminate at electrodes 1682 and 1684, for example, which may be applied to a patient. A device may comprise an output port for connections to more than one pair of electrodes, and claimed subject matter is not limited in this respect.

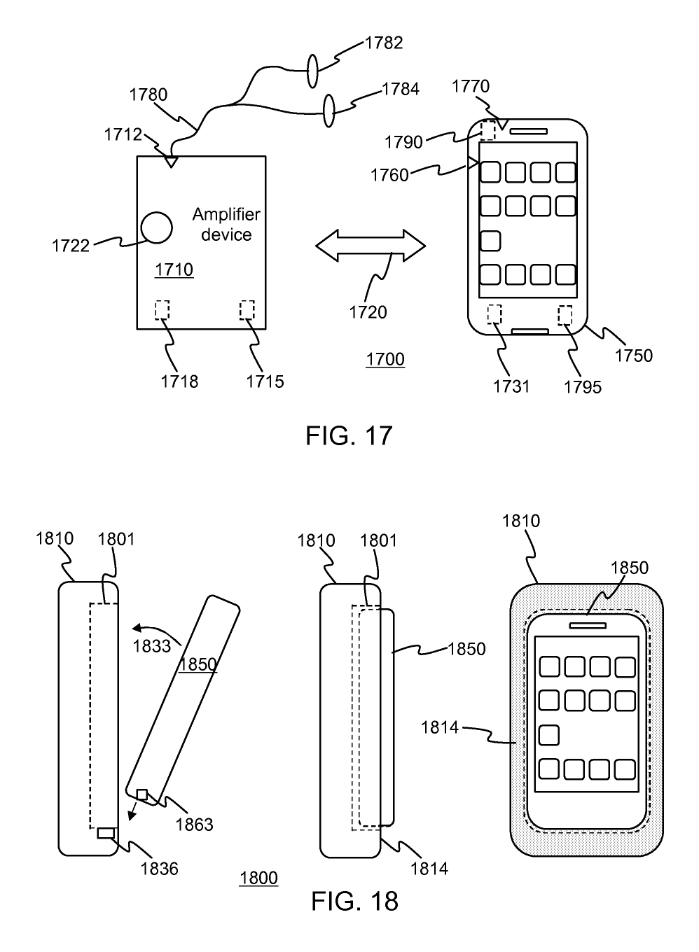

In one implementation, a waveform may comprise a microcurrent waveform. For example, output capability of electronics (e.g., providing an output signal to a headphone jack) of a smartphone may be sufficient to apply hundreds of micro-amps to a patient having an impedance of tens of kilo-ohms. In another implementation, an example of which is shown in FIG. 17, an external amplifier may be used with a smartphone (or other portable electronic device), for example, to amplify relatively small voltage or current amplitudes output by a smartphone to higher values sufficient for application to a patient. Of course, such details of device 1600 are merely examples, and claimed subject matter is not so limited.

FIG. 17 is a schematic diagram of a device 1700 comprising a mobile electronic device 1750 and an amplifier device 1710, according to an embodiment. For example, mobile electronic device 1750 may comprise a smartphone, mobile phone, touch pad, laptop, or other portable (or non-portable) electronic device. In some implementations, desired amplitudes (e.g., current or voltage) of a waveform may be greater than what a mobile electronic device is able to achieve. An external amplifier (e.g., 1710) may be used with a smartphone (or other portable electronic device), for example, to amplify relatively small voltage or current amplitudes of waveforms generated by a smartphone to higher values sufficient for application to a patient. Accordingly, amplifier device 1710 may be used to “boost” signals or waveforms to desirable amplitudes.

In the example embodiment described below, electronic device 1750 is considered to comprise a smartphone for illustrative purposes. Smartphone 1750 may comprise an input port 1760 to receive a waveform file for a waveform to be electrically applied to one or more patients via an output port. For example, an input port may comprise a wireless receiver (e.g., Bluetooth) or a mini- or micro-USB port or other wired connection. In one implementation, smartphone 1750 may wirelessly receive (e.g., from a wireless access point, cellular transmitter, and so on) waveform files via a receiver/transmitter 1790 and stores files in memory 1795, for example.

An output port 1770 may comprise a wireless transmitter (e.g., receiver/transmitter 1790), mini- or micro-USB port or other wired connection, or a headphone jack (e.g., monaural or stereo). Smartphone 1750 may further comprise electronics 1731 configured to generate a waveform having a shape, magnitude, or frequency based, at least in part, on a waveform file. For example, electronics 1731 may comprise a processor configured to execute a waveform file. In an implementation, output port 1770 may provide (e.g., via receiver/transmitter 1790) an electronic signal representative of a waveform to amplifier device 1710. Output port 1770 may provide this signal wirelessly or via cables or conductors, as indicated by arrow 1720. The electronic signal may be generated in smartphone 1750 based, at least in part, on a waveform file executed by a processor (e.g., 1731) running software stored in memory 1795, for example. Amplifier device 1710 may comprise electronics to amplify the electronic signal representative of a waveform of a waveform file executed in smartphone 1750, thereby generating the waveform. The electronics in 1710 may further provide the generated waveform to an output port 1712. An adjustment control 1722 may be used by an operator to adjust amplitude (e.g., voltage or current) of the waveform. A multi-conductor (e.g., stereo) cable 1780 may plug into output port 1712 and terminate at electrodes 1782 and 1784, for example, which may be applied to a patient. A device may comprise an output port for connections to more than one pair of electrodes, and claimed subject matter is not limited in this respect. Of course, such details of device 1700 are merely examples, and claimed subject matter is not so limited.

FIG. 18 includes two schematic side views and a schematic front view of a device 1800 comprising a mobile electronic device 1850 and an amplifier device 1810, according to an embodiment. This embodiment may be similar to that of device 1700, except that mobile electronic device 1850 may be physically mated or electronically connected with amplifier device 1810. Such a configuration may provide a convenience for a user carrying device 1800. For example, such a configuration may allow a user to carry a “single” device as opposed to two separate components, and such a configuration may more securely fit in a user's pocket compared to two separate components. Another benefit may be that connector wires or cables between mobile electronic device 1850 and amplifier device 1810 need not be used. Instead, mobile electronic device 1850 and amplifier device 1810 may be mutually electronically connected via connectors 1863 and 1836, for example.

Mobile electronic device 1850 may be similar to 1750 and may comprise a smartphone, and amplifier device 1810 may have features similar to those of 1710, for example. Mobile electronic device 1850 may include connector 1863. In some implementations, connector 1863 may be used to exchange data with an external device, such as amplifier device 1810 or a computer, for example.

Amplifier device 1810 may include a recessed region 1801 bordered by a raised portion 1814. Recessed region 1801 may have a surface area or dimensions that correspond, at least approximately, to dimensions (e.g., length and width) of mobile electronic device 1850. A depth of recessed region 1801 with respect to a top surface of raised portion 1814 may be less than or greater than a depth of mobile electronic device 1850. For example, the depth of recessed region 1801 may be half the depth of mobile electronic device 1850, though claimed subject matter is not so limited.

As indicated by arrow 1833, mobile electronic device 1850 may be placed into recessed region 1801 of amplifier device 1810 so that mobile electronic device 1850 is retained by amplifier device 1810. Amplifier device 1810 may include connector 1836 to correspond to connector 1863 of mobile electronic device 1850. Electronic signals representative of waveforms may be transferred from mobile electronic device 1850 to amplifier device 1810 via connector 1863, for example. Of course, such details of device 1800 are merely examples, and claimed subject matter is not so limited.

It will, of course, be understood that, although particular embodiments have just been described, claimed subject matter is not limited in scope to a particular embodiment or implementation. For example, one embodiment may be in hardware, such as implemented on a device or combination of devices, for example. Likewise, although claimed subject matter is not limited in scope in this respect, one embodiment may comprise one or more articles, such as a storage medium or storage media that may have stored thereon instructions capable of being executed by a specific or special purpose system or apparatus, for example, to lead to performance of an embodiment of a method in accordance with claimed subject matter, such as one of the embodiments previously described, for example. However, claimed subject matter is, of course, not limited to one of the embodiments described necessarily. Furthermore, a specific or special purpose computing platform may include one or more processing units or processors, one or more input/output devices, such as a display, a keyboard or a mouse, or one or more memories, such as static random access memory, dynamic random access memory, flash memory, or a hard drive, although, again, claimed subject matter is not limited in scope to this example.

The terms, “and” and “or” as used herein may include a variety of meanings that will depend at least in part upon the context in which it is used. Typically, “or” if used to associate a list, such as A, B or C, is intended to mean A, B, and C, here used in the inclusive sense, as well as A, B or C, here used in the exclusive sense. Occasionally, the term “and/or” is also used to associate a list in an inclusive and exclusive sense.

Embodiments described herein may include machines, devices, engines, or apparatuses that operate using digital signals. Such signals may comprise electronic signals, optical signals, electromagnetic signals, or any form of energy that provides information between locations.

In the preceding description, various aspects of claimed subject matter have been described. For purposes of explanation, specific numbers, systems, or configurations may have been set forth to provide a thorough understanding of claimed subject matter. However, it should be apparent to one skilled in the art having the benefit of this disclosure that claimed subject matter may be practiced without those specific details. In other instances, features that would be understood by one of ordinary skill were omitted or simplified so as not to obscure claimed subject matter.

While there has been illustrated and described what are presently considered to be example embodiments, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

What is claimed is:

1. A device comprising:
an input port to receive a waveform file for a waveform from a source external to said device, said waveform to be electrically applied to one or more patients via an output port; and
electronics configured to:
generate said waveform having a shape, magnitude, or frequency based, at least in part, on said waveform file; and
provide said waveform to said output port.

2. The device of claim 1, wherein said electronics are further configured to:
generate said waveform having a shape, magnitude, or frequency based, at least in part, on said waveform file and on patient information regarding said one or more patients, wherein said patient information comprises one or more of: body weight, age, sex, heart condition, injury status, injury history, health history, and history of treatment using said device.

3. The device of claim 1, wherein said waveform file comprises an expirable waveform file that expires after a predetermined time that said waveform is applied to said one or more patients.

4. The device of claim 3, wherein said waveform file further comprises a time code that indicates an expiration time of said expirable waveform file; and said device further comprises:
a clock to measure an elapsed time that said waveform is applied to said one or more patients;
means for determining whether said elapsed time exceeds said expiration time; and
a user interface to indicate if said expirable waveform file is expired.

5. The device of claim 4, wherein said user interface allows a user to enter code to instruct said device to extend said expiration time.

6. The device of claim 1, wherein said input port comprises a universal serial bus (USB) port.

7. The device of claim 1, wherein said user interface comprises a display.

8. The device of claim 1, wherein said device comprises a mobile phone.

9. The device of claim 8, wherein said waveform comprises a microcurrent waveform.

10. A method comprising:
receiving an order from a client for a waveform file comprising code representative of a waveform to be applied by a device to one or more patients; and
electronically transmitting or mailing a digital signal representative of said waveform file via the Internet to said client.

11. The method of claim 10, wherein said waveform file comprises instructions for said device to generate said waveform having a shape, magnitude, or frequency based, at least in part, on one or more characteristics of said one or more patients, wherein said one or more characteristics comprise one or more of: body weight, age, sex, heart condition, injury status, injury history, health history, and history of treatment using said device.

12. The method of claim 10, wherein said waveform file comprises an address code that identifies said client.

13. The method of claim 10, wherein said waveform file comprises a time code that indicates an expiration time of said waveform file.

14. The method of claim 10, wherein said order comprises a renewal order to add time to said expiration time of said waveform file.

15. The method of claim 10, wherein said waveform is approved by a regulatory entity.

16. A method of updating performance characteristics of a device, the method comprising:
placing an order to a provider for a waveform to be electrically applied by a device to one or more patients;
receiving a waveform file comprising digital signals representative of said waveform;
providing said waveform file to said device; and
electrically applying said waveform using said device to said one or more patients.

17. The method of claim 16, wherein said receiving said waveform file comprises receiving said waveform file via the Internet;

18. The method of claim 16, wherein said providing said waveform file to said device further comprises:
storing the received waveform file in a memory device; and
connecting said memory device to said device

19. The method of claim 16, wherein said waveform file comprises an expirable waveform file that expires after a predetermined time that said waveform is applied to said one or more patients.

20. The method of claim 19, wherein said order comprises a renewal order to add time to an expiration time of said expirable waveform file.
